(19) United States
(12) Patent Application Publication
(10) Pub. No.: US 2021/0161797 A1
(43) Pub. Date: Jun. 3, 2021

(54) COSMETIC COMPOSITION COMPRISING A HYPERBRANCHED COPOLYMER AND A HAIR STYLING POLYMER

(71) Applicant: DSM IP ASSETS B.V., Heerlen (NL)

(72) Inventors: Karina HECKER, Kaiseraugst (CH); Mélanie WAECKEL, Kaiseraugst (CH)

(21) Appl. No.: 17/267,909

(22) PCT Filed: Aug. 12, 2019
(86) PCT No.: PCT/EP2019/071588

§ 371 Date: Feb. 11, 2021

(30) Foreign Application Priority Data
Aug. 13, 2018 (EP) 18188737.3

(51) Int. Cl. A61K 8/85; A61K 8/88; A61K 8/81; A61Q 5/06

(52) U.S. Cl. CPC A61K 8/85; A61K 8/88; A61K 8/8176; A61K 8/8182; A61K 8/8147; A61K 8/8152; A61K 2800/594; A61Q 5/06; A61K 2800/544; A61K 2800/5422; A61K 2800/5424; A61K 2800/5426; A61K 2800/5428; A61K 8/8158

(57) ABSTRACT

The present invention relates to a cosmetic composition of a specific hyperbranched copolymer and a hair styling polymer. It has been observed that such a cosmetic composition has a very unique combination of increase of hair elasticity and the maintenance of curl retention of curled hair.

### TECHNICAL FIELD

The present invention relates to the field of cosmetics, particularly to the field of hair care.

### BACKGROUND OF THE INVENTION

Hair care products are an important part of cosmetic products. Due to the different type hairs and the continuous changes in hair styling fashion, the requirements for a specific hair care product vary very much.

However, increase of elasticity of hair and maintaining curl retention of curled hair are consistently highly desired properties in hair care and styling products because these factors are important to prevent the hair from getting frizzy.

“Frizzy” hair is a major hair care problem for consumers, particularly those with coarse, wavy or curly hair, as well as for consumers who have hair which is stressed due to frequently chemical, mechanical or hot iron treatments. Frizzy hair is characterized by a multiplicity of fly-away strands, which gives the total style an unruly look. Frizzy hair is difficult to manage during styling, and it tends to lose its natural shape and/or its curl definition This is a no go for most consumers who desire good style and style retention all day long.

### SUMMARY OF THE INVENTION

The problem to be solved by the present invention is to offer a cosmetic composition which has a very unique combination of increase of hair elasticity and the maintenance of curl retention of curled hair.

Surprisingly, it has been found that a cosmetic composition according to claim 1 comprising a specific hyperbranched copolymer and a hair styling polymer is able to offer a solution for this problem.

Further aspects of the invention are subject of further independent claims. Particularly preferred embodiments are subject of dependent claims.

### DETAILED DESCRIPTION OF THE INVENTION

In a first aspect the present invention relates to a cosmetic composition comprising
a) a hyperbranched copolymer of the monomers
   (i) dodecenyl succinic acid anhydride
   (ii) diisopropanol amine
   (iii) bis-dimethylaminopropyl amine
   having terminal groups of formula

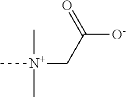

and having a molecular weight Mn of between 1200 and 4000 g/mol; and
b) a hair styling polymer which is either a non-ionic, or anionic or amphoteric hair styling polymer or a cationic hair styling polymer which is selected from the group consisting of polyquaternium-4, polyquaternium-11, polyquaternium-16 and polyquaternium-46.

### Hyper¬Branched Copolymer

The cosmetic composition comprises a hyperbranched copolymer of the monomers
(i) dodecenyl succinic acid anhydride
(ii) diisopropanol amine
(iii) bis-dimethylaminopropyl amine
having terminal groups of formula

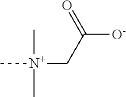

and having a molecular weight Mn of between 1200 and 4000 g/mol.

Said hyperbranched copolymer is preferably prepared by the following steps:
a1) polymerizing the monomers (i) and (ii) and (iii) to yield a polyesteramide having terminal dimethyl amino groups of the formula

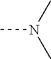

a2) quaternization of the dimethyl amino groups of the polyesteramide of step a1) by 2-chloroacetate, particularly by sodium 2-chloroacetate.

Details for the polymerization step a1) to yield the respective polyesteramide having terminal dimethyl amino groups of the formula

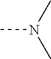

are disclosed for example by EP 2 794 729 B1.

Preferably in the polymerization step a1) the monomer (iii) is added to a mixture of monomers (ii) and (iii) under stirring, followed by heating.

Details of the quaternization step a2) are disclosed as well by EP 2 794 729 B1. Therefore, the entire content of EP 2 794 729 B1 is hereby incorporated by reference.

The term “molecular weight Mn” stands for the number average molecular weight (regularly called also number average molar mass).

The amount of the hyperbranched copolymer of the monomers
(i) dodecenyl succinic acid anhydride
(ii) diisopropanol amine
(iii) bis-dimethylaminopropyl amine
having terminal groups of formula

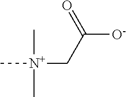

and having a molecular weight Mn of between 1200 and 4000 g/mol.

is typically between 0.1 and 2% by weight, relative the weight of the cosmetic composition.

It is preferred that the molar ratio of the monomers (i) to (ii) is between 5:1 and 0.5:1, particularly between 4:1 and 1:1, preferably between 3:1 and 3:2.

It is further preferred that the molar ratio of the monomers (i) to (iii) is between 5:1 and 0.5:1, particularly between 3:1 and 1:1, preferably between 2.5:1 and 1.1:1.

The hyperbranched copolymer has preferably a number average molecular weight Mₙ of between 1400 and 3000 g/mol, preferably between 2100 and 2400 g/mol, more preferably between 2100 and 2300 g/mol.

Preferably, the hyperbranched polymer is polyquaternium-110, also identified by CAS Number 1323977-82-7.

### Hair Styling Polymer

The cosmetic composition comprises, furthermore, an additional polymer which is a hair styling polymer which is either a non-ionic, or anionic or amphoteric hair styling polymer or a cationic hair styling polymer which is selected from the group consisting of polyquaternium-4, polyquaternium-11, polyquaternium-16 and polyquaternium-46. Hair styling polymers per se are known to the person skilled in the art.

Examples of non-ionic hair styling polymers are homopolymers of N-vinylpyrrolidone and copolymers of N-vinylpyrrolidone (NVP) with compatible nonionic monomers such as vinyl acetate (VA). Nonionic polymers containing N-vinylpyrrolidone in various weight average molecular weights are commercially available, for example from Ashland. Specific examples of such materials are homopolymers of N-vinylpyrrolidone having an average molecular weight of about 630,000 sold under the name PVP K-90 and are homopolymers of N-vinylpyrrolidone having an average molecular weight of about 1,000,000 sold under the name of PVP K-120. Examples of copolymers of N-vinylpyrrolidone and vinyl acetate in different ratios of N-vinylpyrrolidone:vinyl acetate such as 70:30, 60:40, 50:50, 30:70 are commercially available. They are sold for example under tradenames comprising the identifier VA-64 (for ratio NVP/VA=60/40) or VA-55 (for ratio NVP/VA=50/50) or VA-37 (for ratio NVP/VA=30/70) or VA-73 (for ratio NVP/VA=70/30) from different suppliers, such as Ashland or BASF.

Preferred non-ionic hair styling polymers are PVPs (Polyvinylpyrrolidones) and N-vinylpyrrolidone/vinylacetate Copolymers, preferably in a ratio of NVP/VA=60/40.

Preferred cationic hair styling polymers are polyquaternium-16 and polyquaternium-46.

Preferred amphoteric hair styling polymers. Particularly preferred are octylacrylamide/acrylate/butylaminoethyl methacrylate copolymers. Examples for such polymers are commercially available under the trademark AMPHOMER® Polymers from Nouryon.

Examples of anionic hair styling polymers are: copolymers of vinyl acetate and crotonic acid; terpolymers of vinyl acetate, crotonic acid and a vinyl ester of an alpha-branched saturated aliphatic monocarboxylic acid such as vinyl neo-decanoate; copolymers of methyl vinyl ether and maleic anhydride (molar ratio about 1:1) wherein such copolymers are 50% esterified with a saturated alcohol containing from 1 to 4 carbon atoms such as ethanol or butanol; acrylic copolymers containing acrylic acid or methacrylic acid as the anionic radical-containing moiety with other monomers such as: esters of acrylic or methacrylic acid with one or more saturated alcohols having from 1 to 22 carbon atoms (such as methyl methacrylate, ethyl acrylate, ethyl methacrylate, n-butyl acrylate, t-butyl acrylate, t-butyl methacrylate, n-butyl methacrylate, n-hexyl acrylate, n-octyl acrylate, lauryl methacrylate and behenyl acrylate); glycols having from 1 to 6 carbon atoms (such as hydroxypropyl methacrylate and hydroxyethyl acrylate); styrene; vinyl caprolactam; vinyl acetate; acrylamide; alkyl acrylamides and methacrylamides having 1 to 8 carbon atoms in the alkyl group (such as methacrylamide, t-butyl acrylamide and n-octyl acrylamide); and other compatible unsaturated monomers.

Other suitable anionic hair styling polymers include carboxylated polyurethanes. Carboxylated polyurethane resins are linear, hydroxyl-terminated copolymers having pendant carboxyl groups. They may be ethoxylated and/or propoxylated at least at one terminal end. The carboxyl group can be a carboxylic acid group or an ester group, wherein the alkyl moiety of the ester group contains one to three carbon atoms. The carboxylated polyurethane resin can also be a copolymer of polyvinylpyrrolidone and a polyurethane, having a CTFA designation PVP/polycarbamyl polyglycol ester. Suitable carboxylated polyurethane resins are disclosed in EP-A-0619111 and U.S. Pat. No. 5,000,955. Other suitable hydrophilic polyurethanes are disclosed in U.S. Pat. Nos. 3,822,238; 4,156,066; 4,156,067; 4,255,550 and 4,743,673.

Preferred anionic hair styling polymers are anionic (meth)acrylate copolymers, particularly copolymers of at least two monomers selected from the group consisting of acrylic acid, methacrylic acid, C₁₋₆-alkylester of acrylic acid, C₁₋₆-alkylester of methacrylic acid, C₂₋₁₀-aminoalkylester of acrylic acid, C₂₋₁₀-aminoalkylester of methacrylic acid, C₁₋₆-alkylamides of acrylic acid and C₁₋₆-alkylamides of methacrylic acid.

In the most preferred embodiment, the hair styling polymer is an anionic acrylic copolymer, particularly an acrylic copolymer of the monomers methacrylic acid, n-butyl methacrylate, ethyl acrylate and ethyl methacrylate. Preferred are those acrylic copolymers for example disclosed in WO2011/057882A1. Details to said polymers and their production can be also found in WO2011/057882A1. Particularly preferred as hair styling polymer is the butyl methacrylate/methacrylic acid/ethyl acrylate/ethyl methacrylate copolymer commercially available from DSM Nutritional Products Ltd under the trademark TILAMAR® Fix A1000 [CAS: 1070166-98-1].

It has been observed that using the above acrylic copolymer in the cosmetic composition creates a particular good and natural hair feel.

The weight ratio of hyperbranched copolymer and hair styling polymer is typically between 0.1:1 and 0.8:1, preferably between 0.2:1 and 0.7:1, more preferably between 0.23:1 and 0.65:1.

The amount of the hyperbranched copolymer in the cosmetic composition is typically between 0.1 and 10, preferably between 0.5 and 7, more preferably between 1 and 3% by weight.

### Further Ingredients

The cosmetic compositions can obtain further ingredients principally known by the person skilled in the art.

Particularly, the cosmetic composition comprises water. The amount of depends strongly on the type of application and use of the composition.

The cosmetic composition may contain further ingredients to enhance the performance and/or consumer acceptability such as antioxidants, thickeners, softeners, antifoaming agents, moisturizers, fragrances, co-surfactants, fillers, sequestering agents, cationic-, nonionic- or amphoteric polymers or mixtures thereof, acidifying or basifying agents, dyes, colorants, pigments or nanopigments, pearlizers or opacifiers, organic or inorganic particles, viscosity modifiers, and natural hair nutrients such as botanicals, fruit extracts, sugar derivatives and/or amino acids or any other ingredients usually formulated into the cosmetic compositions. The necessary amounts of the adjuvants and additives can, based on the desired product, easily be chosen by a person skilled in the art in this field and will be illustrated in the examples, without being limited hereto.

Particularly, the cosmetic composition may comprise further surfactants. In one embodiment the cosmetic composition comprises sulfate surfactants, i.e. alkyl sulfates, alkyl ether sulfates, alkyl amido ether sulfates, alkylaryl polyether sulfates or monoglycerides sulfate

In another, preferred, embodiment the cosmetic composition comprises sulfate-free surfactants, particularly cationic, anionic, non-ionic or amphoteric surfactants. Examples of suitable anionic surfactants are the alkanoyl isothionates, alkyl succinates, alkyl sulphosuccinates, alkyl ether sulphosuccinates, N-alkyl sarcosinates, alkyl phosphates, alkyl ether phosphates, and alkyl ether carboxylic acids and salts thereof, especially their sodium, magnesium, ammonium or mono-, di- or triethanolamine salts. The alkyl and acyl groups generally contain from 8 to 18, preferably from 10 to 16, carbon atoms and may be unsaturated, alkyl ether sulphosuccinates, alkyl ether phosphates and alkyl ether carboxylic acids and salts thereof may contain from 1 to 20 ethylene oxide or propylene oxide units per molecule.

In particular, the anionic surfactants are selected from sodium oleyl succinate, ammonium lauryl sulphosuccinate, sodium lauryl ether sulphosuccinate, sodium cocoyl isethionate, sodium lauryl isethionate, lauryl ether carboxylic acid and sodium N-lauryl sarcosinate or mixtures thereof. Preferred anionic surfactants are sodium lauryl ether sulphosuccinate (n) EO, (where n is from 1 to 4, in particular n is 3).

Examples of nonionic surfactants include condensation products of aliphatic (C8-C18) primary or secondary linear or branched chain alcohols with alkylene oxides, usually ethylene oxide and generally having from 6 to 30 ethylene oxide groups. Other representative nonionic surfactants include mono- or di-alkyl alkanolamides such as e.g. coco mono- or di-ethanolamide and coco mono-isopropanolamide. Further nonionic surfactants which can be included in the cosmetic compositions are the alkyl polyglycosides (APGs). Typically, the APG is one which comprises an alkyl group connected (optionally via a bridging group) to a block of one or more glycosyl groups such as e.g. Oramix™ NS 10 ex Seppic; PLANTACARE® 818UP, PLANTACARE® 1200 and PLANTACARE® 2000 ex BASF.

Examples of amphoteric (or zwitterionic) surfactants are alkyl amine oxides, alkyl betaines, alkyl amidopropyl betaines, alkyl sulphobetaines (sultaines), alkyl glycinates, alkyl carboxyglycinates, alkyl amphoacetates, alkyl amphopropionates, alkylamphoglycinates, alkyl amidopropyl hydroxysultaines, acyl taurates and acyl glutamates, wherein the alkyl and acyl groups have from 8 to 19 carbon atoms. Typical amphoteric and zwitterionic surfactants for use in the cosmetic compositions include lauryl amine oxide, cocodimethyl sulphopropyl betaine, lauryl betaine, cocamidopropyl betaine (CAPB), sodium cocoamphoacetate and disodium cocoamphodiacetate. Particularly preferred amphoteric or zwitterionic surfactants which can be used in the cosmetic composition are cocamidopropyl betaine, cocoamphoacetate or cocoamphodiacetate such as most preferably sodium cocoamphoacetate.

Mixtures of any of the foregoing amphoteric or zwitterionic surfactants may also be suitable. Preferred mixtures are those of cocamidopropyl betaine with further amphoteric or zwitterionic surfactants as described above such as in particular with sodium cocoamphoacetate or disodium cocoamphodiacetate.

The cosmetic composition may further comprise a suspending agent. Suitable suspending agents are selected from polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives. The long chain acyl derivative is desirably selected from ethylene glycol stearate, alkanolamides of fatty acids having from 16 to 22 carbon atoms and mixtures thereof. Ethylene glycol distearate and polyethylene glycol 3 distearate are preferred long chain acyl derivatives, since these impart pearlescence to the cosmetic composition. Polyacrylic acid is available commercially as Carbopol® 420, Carbopol® 488 or Carbopol® 493. Polymers of acrylic acid cross-linked with a polyfunctional agent may also be used; they are available commercially as Carbopol® 910, Carbopol® 934, Carbopol® 941, Carbopol® 980 and Carbopol® Ultrez 10 Polymer. Examples of suitable copolymers of a carboxylic acid containing monomer and acrylic acid esters are Carbopol® 1342, Carbopol® Ultrez 20 or Carbopol® Ultrez 21, Pemulen TR1 or Pemulen TR2. All Carbopol® or Pemulen® materials are available from Lubrizol.

A suitable heteropolysaccharide gum is xanthan gum, for example Keltrol®-types or Kelzan®-types from Kelco, Vanzan NF from RT Vanderbilt Inc. or Rhodicare®-types from Rhodia.

Mixtures of any of the above suspending agents may be used. Preferred is a mixture of cross-linked polymer of acrylic acid and crystalline long chain acyl derivative.

The cosmetic composition may further contain neutralizers, particularly aminomethyl propanol, for example AMP-Ultra PC, commercialized from ANGUS Chemical Company.

The cosmetic composition as described above is particularly a hair spray, cream or gel.

It is preferred if the cosmetic compositions are left on hair after application and not immediately washed off (within 30 minutes of application). Such products are called “leave on” formulations. Preferred product forms are leave on formulations such as gels, mousses, sprays and aerosols. It is preferable if the pH at 30° C. of the composition is from 2.5 to 9, more preferably above 4, most preferably from 5 to 8. The cosmetic compositions of the present invention can comprise a carrier, or a mixture of such carriers, particularly which are suitable for application to the hair. The carriers are preferably present from 0.5% to 99.5%, more preferably from 5.0% to 99.5%, even more preferably from 10.0% to 98.0%, of the composition. As used herein, the phrase “suitable for application to hair” means that the carrier does not damage or negatively affect the aesthetics of hair or cause irritation to the underlying skin. Preferably the cosmetic compositions comprise a buffer or pH adjuster. Preferred buffers or pH adjusters include weak acids and bases such glycine/sodium hydroxide, citric acid, lactic acid, succinic acid, acetic salt and salts thereof. Frequently a mixture of buffering system is used such as sodium citrate and citric acid. Carriers suitable for use with cosmetic compositions include, for example, those used in the formulation of hair sprays, mousses, tonics, waters, creams gels, shampoos, conditioners, and rinses. The choice of appropriate carrier will depend on the particular product to be formulated. The carriers used herein can include a wide range of components conventionally used in cosmetic compositions. Mousses and aerosol hair sprays can also utilize any of the conventional propellants to deliver the material as a foam (in the case of a mousse) or as a fine, uniform spray (in the case of an aerosol hair spray). Examples of suitable propellants include materials such as trichlorofluoromethane, dichlorodifluoromethane, difluoroethane, dimethylether, propane, n-butane or isobutane. The level of propellant can be adjusted as desired but is generally from about 3% to about 30% by weight based on total weight for mousse compositions and from about 15% to about 50% by weight based on total weight for aerosol hair spray compositions.

Hair styling creams or gels also typically contain a structurant or thickener, typically in an amount of from 0.01% to 10% by weight. Suitable spray containers are well known in the art and include conventional, non-aerosol pump sprays i.e., “atomisers”, aerosol containers or cans having propellant, as described above, and also pump aerosol containers utilizing compressed air as the propellant.

The cosmetic may include conditioning materials such as surfactants, cationic conditioners suitable for hair, quaternary silicone polymers, silicone based conditioners and their emulsions, and amino functional silicones and their emulsions. Further general ingredients suitable for cosmetic compositions include, carboxylic acid polymer thickeners for hair shampoo and conditioner compositions.

It has been found that the cosmetic composition as described above in great detail has a very unique combination of increasing of the hair elasticity and maintaining the curl retention of curled hair.

The hair elasticity describes the ability of a hair swatch to return to its original shape after mechanical deformation, e.g. how good a curled swatch jumps back into shape after stretching or a straight swatch jumps back after curling. The hair elasticity is typically assessed by measurements of hair strands by a mechanical bending test.

Pre-washed hair swatches are treated with the product of invention, combed 5 times and dried in oven at 40 min at 45° C. After storage overnight under controlled humidity and temperature (21° C., 65% rH (=relative humidity)), the bending forces measurements are performed 3 times successively. First bending force determines the maximum bending force needed to break the “fixative/hold-providing” film. “Hysteresis” bending forces are measured by bending once, return to initial start point and bend again (up to 2 times) to evaluate the flexibility of the hold. Details can be found in “Substantiating Claims for Hair Care Products”, M. Brandt et al., SOFW 2016, 142, 17-24.

The curl retention describes the hold of hair curls at prolonged exposure to high humidity. In this method, curls are made by treating pre-washed hair swatches with product of invention, combed 5 times and rolling them around a curler. When the swatches are dried (40 min., 45° C.) and stored overnight under controlled humidity and temperature (21° C., 65% rH), the curler is carefully removed and the curled swatches are placed in a climatic chamber at high relative humidity (21° C., 90% rH). The length of the curl is then measured at set time periods up to 6 hours and the curl retention (CR) is calculated in relation to the original length of the curl. Details can be found in “Substantiating Claims for Hair Care Products”, M. Brandt et al., SOFW 2016, 142, 17-24.

The cosmetic combinations maintain the curl retention (CR). The term “maintaining” as used in this document is used to mean that the curl retention is not less than 45%, preferably not less than 50%, more preferably not less than 55%, after 360 minutes in the climatic chamber measured using the above method.

Hence, in a further aspect, the invention relates to the use of a cosmetic combination as described above in great detail for increasing of the hair elasticity and/or maintaining the curl retention (CR) of curled hair.

### EXAMPLES

The present invention is further illustrated by the following experiments. These examples are illustrative only and are not intended to limit the scope of the invention in any way.

### Preparation of Hyperbranched Copolymer (HBC1)

The hyperbranched copolymer HBC1 of the monomers dodecenyl succinic acid anhydride and diisopropanol amine and bis-dimethylaminopropyl amine has been prepared according to example 3 in EP 2 794 729 B1 using 237.59 g of N,N-bis(N′N′-dimethylaminopropyl)amine and 112.6 g diisopropanol amine and 426.89 g of dodecenylsuccinic anhydride. After heating and vacuum, the residual carboxylic acid content of <0.3 meq/g (tritrimetrical analysis) AV=9.8 mg KOH/g and amine content of 2.99 meq/g (tritrimetrical analysis) and a molecular weight Mn=2240 Da was obtained. This product has been reacted with sodium chloroacetate in water and stirred at 80° C. until ¹H-NMR analysis shows a complete conversion of the chloroacetate to obtain the hyperbranched copolymer HBC1 which has terminal groups of the formula

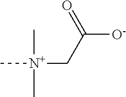

and a molecular weight Mn of 2.3 kDa.

The hyperbranched copolymer HBC1 was used as a 45% solution in water in the following experiments.

### Compositions

For the experiments, the compositions 1, 2, and 3, respectively Ref.1 (without HBC1) have been prepared according to the amounts given in table 1.

TABLE 1
Compositions

| | Ref. 1 | 1 | 2 | 3 |
|---|---|---|---|---|
| Ethanol [wt.-%] | 55 | 55 | 55 | 55 |
| AMP-Ultra PC¹[wt.-%] | 1.07 | 1.07 | 1.07 | 1.07 |
| TILAMAR ® Fix A 1000² | 5 | 5 | 5 | 5 |
| [wt.-%] | | | | |
| HPC1³ [wt.-%] | 0 | 2.25 | 4.45 | 6.65 |
| Water [wt.-%] | at 100 | at 100 | at 100 | at 100 |

¹aminomethyl propanol, from ANGUS Chemical Company.
²from DSM Nutritional Products Ltd.
³polymer content 45% by weight

### Assessment of Elasticity

The elasticity, respectively the stiffness, has been assessed by conducting a 3-point bending stiffness test.

Five hair swatches were used per test product (Kerling Art.826200 European hair, weight: 0.7 g+/−0.05 g, length 12 cm, Euro Natur, Color 6/0). Bending stiffness is measured 3× successively for each hair swatch

The hair swatches have been pre-washed twice with standard cleansing shampoo: 0.25 ml/g hair, foaming for 30 seconds, rinsing with 38° C. warm water for 30 seconds at 5 l/min, and then dried overnight in a climate room at 21° C./65% rel. humidity.

Each swatch was treated with 0.3 g of the respective composition of table 1. The product is evenly applied with a syringe from root to tip, combed 5 times, dried for 40 min at 45° C. and stored at least for 12 hours at 21° C./65% rel. humidity.

Then the elasticity, respectively the stiffness, has been assessed by conducting a 3-point bending stiffness test performed on a Texture Analyzer (TA XTplus). The force which is applied is monitored and plotted against the bending distance starting distance (d₀). The first relevant point is the maximal force (Fₘₐₓ) measured at a distance (dₘₐₓ). This force relates to the breaking of the polymer film. Now the force is released by reducing the bending distance. In a further step the bending distance is increased again to distance (dₘₐₓ) and the force (F₁) is measured. Now the force is released again by reducing the bending distance. Finally, the bending distance is increased again to distance (dₘₐₓ) and the force (F₂) is measured.

In table 2, the measured forces are compiled.

TABLE 2
3-point bending stiffness test.

| | Ref. 1 | 1 | 2 | 3 |
|---|---|---|---|---|
| Fₘₐₓ [g] | 137.5 | 142.8 | 144.9 | 149.4 |
| F₁ [g] | 107.7 | 96.7 | 86.3 | 74.9 |
| F₂ [g] | 99.4 | 82.9 | 71.0 | 65.7 |

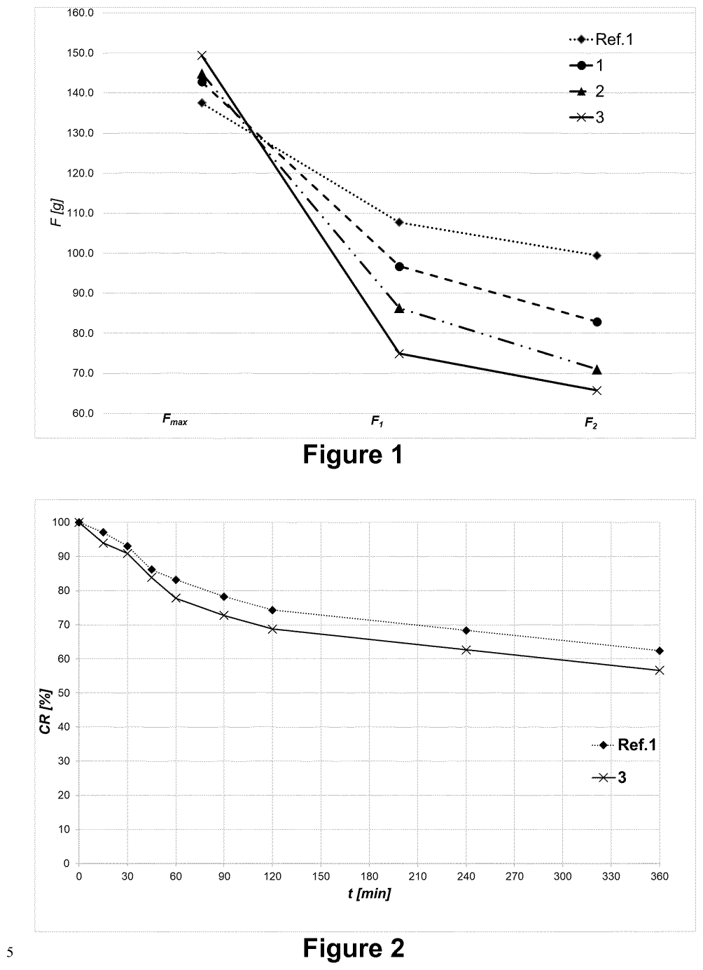

FIG. 1 is a graphic representation of these values. The results given in table 2 and FIG. 1 show, on the one hand, that the compositions/and 2 and 3 have a higher force Fₘₐₓ, which indicates a better hold, respectively a better “styling hold”, as compared to the reference composition Ref.1. On the other hand, the compositions/and 2 and 3 have a significant lower force (F₁) and force (F₂), which indicates a better elasticity, as compared to the reference composition Ref.1.

It further shows that these advantages are increased by an increase of the hyperbranched polymer of the monomers (i), (ii) and (iii).

### Assessment of Curl Retention

Three hair swatches were used per test product (Kerling Art.826500, KT dicht, length 23 cm, free hair 21 cm, Euro Natur, Color 6/0).

The hair swatches have been pre-washed with a standard shampoo (10 seconds wetting, 30 seconds shampooing, 30 seconds rinsing), combed 5 times and dried overnight under defined conditions (21±1° C., 65% rH).

Then each swatch was treated with 0.5 ml of the compositions Ref.1 or 3 of table 1. The product is evenly applied with a syringe from root to tip, combed 5 times. The hair swatches have been wound on a curler (12 mm diameter, plastic wire) and dried in oven for 40 min. at 45° C.

After conditioning over night at 21° C. and 65% rH, the hair swatches are removed from the curler and are suspended in specific climate chamber (21° C., 90% rH) for up to 6 hours. The length of the curl has been measured at the very beginning as original length of the curl (L₀). The length of the curl height has been monitored over time in these conditions (Lₜ) for a time up to 360 minutes. The curl retention (=CR=Lₜ/L₀) as a function of time has been calculated and indicated in table 3 and shown in FIG. 2. The higher this value the better is the better the curl retention is.

TABLE 3
Curl retention values.

| Curl retention (CR) [%] | Ref. 1 | 3 |
|---|---|---|
| After 0 minutes | 100 | 100 |
| After 15 minutes | 97.0 | 93.9 |
| After 30 minutes | 93.1 | 90.9 |
| After 45 minutes | 86.2 | 83.8 |
| After 60 minutes | 83.2 | 77.8 |
| After 90 minutes | 78.2 | 72.7 |
| After 120 minutes | 74.3 | 68.7 |
| After 240 minutes | 68.3 | 62.6 |
| After 360 minutes | 62.4 | 56.6 |

The results given in table 3 and FIG. 2 show that the excellent curl retention of the composition comprising the styling polymer (Ref.1) is maintained when the composition comprises additionally also a hyperbranched copolymer of monomers (i) and (ii) and (iii) (3).

Hence, an excellent curl retention of curled hair is observed in combination of an excellent elasticity.

1. A cosmetic composition comprising
a) a hyperbranched copolymer of the monomers
(i) dodecenyl succinic acid anhydride
(ii) diisopropanol amine
(iii) bis-dimethylaminopropyl amine
having terminal groups of formula 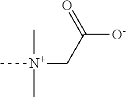
and having a molecular weight Mn of between 1200 and 4000 g/mol; and
b) a hair styling polymer which is either a non-ionic, or anionic or amphoteric hair styling polymer or a cationic hair styling polymer which is selected from the group consisting of polyquaternium-4, polyquaternium-11, polyquaternium-16 and polyquaternium-46.

2. The composition according to claim 1 wherein the hyperbranched copolymer is prepared by steps
a1) polymerizing the monomers (i) and (ii) and (iii) to yield a polyesteramide having terminal dimethylamino group of the formula 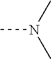
a2) quaternization of the dimethyl amino groups of the polyesteramide of step a1) by 2-chloroacetate, particularly by sodium 2-chloroacetate.

3. The cosmetic composition according to claim 1 wherein the molar ratio of the monomers (i) and (ii) is between 5:1 and 0.5:1, particularly between 4:1 and 1:1, preferably between 3:1 and 3:2.

4. The cosmetic composition according to claim 1 wherein the molar ratio of the monomers (i) and (iii) is between 5:1 and 0.5:1, particularly between 3:1 and 1:1, preferably between 2.5:1 and 1.1:1.

5. The cosmetic composition according to claim 1 wherein the hair styling polymer is
a non-ionic hair styling polymer selected from the group consisting of homopolymers of N-vinylpyrrolidone (=polyvinylpyrrolidones) and copolymers of N-vinylpyrrolidone and vinylacetate;
or
a cationic hair styling polymer selected from the group consisting of polyquaternium-4, polyquaternium-11, polyquaternium-16 and polyquaternium-46;
or
an anionic hair styling polymer selected from the group consisting of anionic (meth)acrylate copolymers;
or
an amphoteric hair styling polymer selected from the group consisting of octylacrylamide/acrylate/butylaminoethyl methacrylate copolymers.

6. The cosmetic composition according to claim 1 wherein the hair styling polymer is an acrylic copolymer of the monomers methacrylic acid, n-butyl methacrylate, ethyl acrylate and ethyl methacrylate.

7. The cosmetic composition according to claim 1 wherein the hyperbranched copolymer has a number average molecular weight Mₙ of between 1400 and 3000 g/mol, preferably between 2100 and 2400 g/mol.

8. The cosmetic composition according to claim 1 wherein the hyperbranched copolymer is polyquaternium-110.

9. The cosmetic composition according to claim 1 wherein the weight ratio of hyperbranched copolymer and hair styling polymer is between 0.1:1 and 0.8:1, preferably between 0.2:1 and 0.7:1, more preferably between 0.23:1 and 0.65:1.

10. The cosmetic composition according to claim 1 wherein the amount of the hyperbranched copolymer in the cosmetic composition is between 0.1 and 10, preferably between 0.5 and 7, more preferably between 1 and 3% by weight.

11. Use of a cosmetic combination according to claim 1 for increasing of the hair elasticity and/or maintaining the curl retention of curled hair.
